(19) United States
(12) Patent Application Publication
(10) Pub. No.: US 2007/0021657 A1
(43) Pub. Date: Jan. 25, 2007

(54) System and method for enhanced medicament-based treatment of disease

(75) Inventors: Oscar Enrique Garcia Galvez, Guatemala City (GT); Marcus Augusto Ximenes Diniz, Sao Paulo (BR)

Correspondence Address: SYNNESTVEDT & LECHNER, LLP, 2600 ARAMARK TOWER, 1101 MARKET STREET, PHILADELPHIA, PA, 191072950, US

(73) Assignee: Aventis Pharma Ltda., Edifico Altanta Sao Paulo (BR)

(21) Appl. No.: 10/555,198

(22) PCT Filed: Apr. 27, 2004
(86) PCT No.: PCT/IB04/02327

§ 371 Date: Oct. 28, 2005

Related U.S. Application Data
(60) Provisional application No. 60/467,082, filed on May 1, 2003.

(30) Foreign Application Priority Data
Sep. 29, 2003 (GB) 0322661.0

(51) Int. Cl. A61B 5/00; G06Q 10/00

(52) U.S. Cl. 600/300; 705/2; 128/920

(57) ABSTRACT

The present invention relates to a method where one acquires and uses data concerning a disease or health dysfunction, providing by way of its processing into useful information an optimized treatment for such a disease or health dysfunction. In another aspect of the invention, the use of such a method leads to an increase in the sales of the medicament for the treatment of such a disease or health dysfunction.

### FIELD OF THE INVENTION

The present invention relates generally to computerized data acquisition and analysis, and more particularly to an interactive system and method for broad-based data acquisition and analysis for developing enhanced patient treatment plans, which leads to increased medicament sales.

### DISCUSSION OF RELATED ART

The treatment of diseases and health dysfunctions (collectively, “disease”) relies heavily on the intake of medicaments. Such medicaments are frequently the result of extensive and extremely expensive research and development by pharmaceutical companies. Further costs are incurred in testing, clinical trials, seeking FDA or similar approval, etc. (collectively, “testing”). Such testing results in the gathering of information that physicians rely upon when selecting an appropriate medicament and prescribing a treatment regimen for a given patient having a given disease.

However, such testing is limited in time and scope. Therefore, regardless of the money and effort invested in the development of a medicament, there are always conditions and situations that could not be actually tested, as every human or other living organism is unique in its response to a foreign chemical substance. Additionally, many endogenous and exogenous patient and environmental factors may affect a medicinal treatment, such as: inherited genetics; concurrent diseases; eating, drinking and exercising habits; healthy life habits; personal hygiene; employment, family or other stress factors; temperature, pressure, humidity or other climatic aspects; air or sound pollution; natural or acquired physical posture, etc.

Accordingly, while the available information is potentially beneficial to the physician and humans generally, such information is typically not sufficiently extensive, and/or has limited relevance to a particular patient, and so may prevent selection of medicaments and treatment plans most beneficial to that particular patient.

In addition to the patient and environmental factors discussed above, commercial factors strongly influence the selection and prescribing of medicaments, and may prevent selection of medicaments and treatment plans most beneficial to the patient. For example, such factors include: a cost/benefit ratio; the extent of exposure of a certain brand of medicament in the appropriate media; the familiarity of a prescribing physician with that medicament; the advantages and disadvantages of a medicament relative to a competing product; the easy access and quick response one obtains from the manufacturer or creator of the medicament, etc.

All of these factors complicate the tasks of educating the relevant medical professionals regarding a newly developed medicament, and ensuring proper treatment of patients having a disease or dysfunction treatable by the medicament.

We, as the inventors hereof, have recognized a need for ways to enhance treatment of diseases, while improving sales of beneficial medicaments.

While related generally to medicaments and/or the use of an information network, the following documents do not disclose, suggest or in any way foresee the present invention: WO0154027, WO0159570, WO0186506, WO0205187, EP1244047, WO0159570, WO0223440, JP2001338055, JP2002132890, WO0146888, WO0127838, WO0118726, US20020143578, U.S. Pat. No. 6,322,504, US20020082906, US20020147135, the disclosures of each of which are hereby incorporated herein by reference.

### SUMMARY

The present invention provides a system and method for enhancing medicament-based treatment of disease by acquiring and analyzing patient and patient treatment data from the general population of consumers. In a preferred embodiment, such data is gathered after any testing, FDA or other government approvals, etc. are complete, such that the general population of consumers may be used to provide further research information and feedback, without compromising in any way the usual medicament testing and approval processes, and therefore without causing or increasing any health risks to consumers. Conceptually, the present invention allows for real-time, post-FDA or other approval continued medicament research and/or feedback using a much larger sample size (participating members of the general population) than typical preliminary/pre-approval testing, and use of resulting information to enhance treatment of patients and increase medicament sales.

More specifically, the acquired data is used to identify information that is useful in prescribing an enhanced treatment for the disease. Advantageously, the present invention provides the capability to gather such useful information from data that already exists, but would otherwise remain unreported or unevaluated, e.g. individual patient medical records. In particular, the present invention allows for reporting of such data to a centralized repository to allow for statistical or other analysis in an aggregated fashion. Such analysis allows for consideration with a considerably larger sample size, and to consider a greater number of factors, than typically considered in preliminary testing or clinical trials. It further allows for investigations of various factors over time by allowing altering of an online or other questionnaire for gathering data over time, as desired. This provides for better information, with a greater level of detail, for consideration in selecting a medicament and prescribing a treatment plan, with a more beneficial effect to patents having a given disease.

Such information is made available to interested parties, such as patients, potential patients, physicians, and other members of the medical and/or research communities so that the availability of such information is not only helpful to patients, but also lends a greater level of confidence and/or awareness to physicians in prescribing the medicament, which results in increased sales of a medicament for which the data acquisition method is utilized.

### BRIEF DESCRIPTION OF THE DRAWINGS

The present invention will now be described by way of example with reference to the following drawings in which:

FIG. 1 is a block diagram of an exemplary system and communications network environment in which the invention may be employed;

FIG. 2 is a flow diagram illustrating an overview of an exemplary method for enhanced medicament-based treatment of disease in accordance with an exemplary embodiment of the present invention;

FIG. 3 is a flow diagram illustrating an aspect of the exemplary method of FIG. 2; and

FIG. 4 is a block diagram of a server of the system of FIG. 1.

### DETAILED DESCRIPTION

In one aspect, the present invention provides a method for enhanced medicament-based treatment of disease whereby data relating to patients taking a medicament for treatment of a disease is acquired from diverse sources, analyzed in aggregated form, and used to identify information useful in treating the disease, e.g. by identifying correlations between patient characteristics and treatment parameters, by identifying areas for further research or investigation, and by using such information to develop treatment guidelines that can be communicated back to patients, treating physicians, etc. to enhance the treatment of the patients suffering the disease.

In another aspect, the present invention provides for use of such information to educate medical professions and provide them with a greater level of confidence in prescribing the medicament and/or a greater level of ability to determine when prescribing of the medicament is advisable, which results in increased sales of a medicament for which the inventive method is utilized.

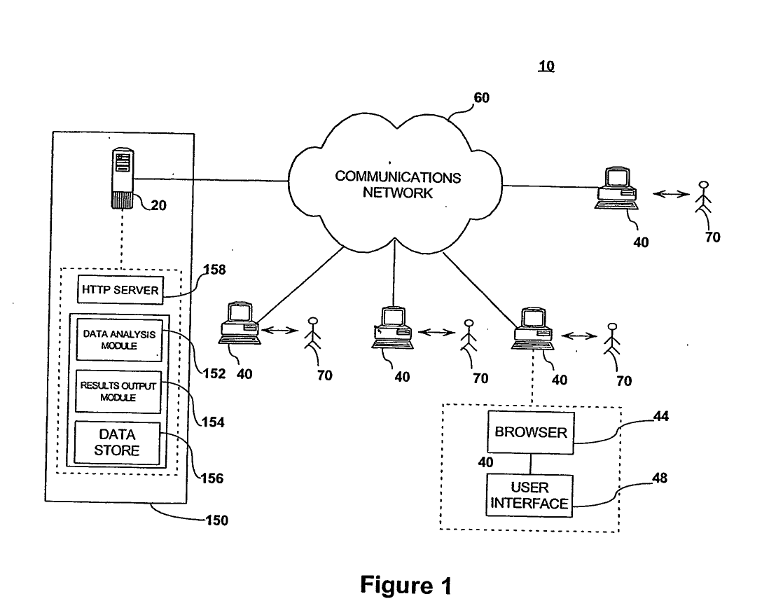

The data acquisition method of the present invention may be understood with reference to the exemplary, simplified network environment 10 of FIG. 1. The network environment 10 may be used with a data processing system 150 for enhancing medicament-based treatment of disease, although it should be understood that the invention need not be employed in such a network environment 10. As shown in FIG. 1, a server 20 of the system 150 can communicate with one or more client computers 40 via a computer communications network 60. By way of example, the communications network 60 could be a local area network (LAN), wide area network (WAN), an intranet, the Internet, etc. Any network configuration allowing data to flow between points of access may be used. In this example, the communications network 60 is the Internet, and server 20 is an HTTP web server. Accordingly, the server 20 and client computers 40 can communicate with each other via a common protocol, such as the hypertext transfer protocol (HTTP), as well known in the art. It will be understood by those skilled in the art that an actual network topology may include numerous clients and servers.

The client computer 40 interfaces with communications network 60 via web browser software 44 stored and/or running on the client computer 40 to allow a member of the general public, a physician, a nurse or other health care provider, etc. (collectively, a “user”) 70 to access web sites, etc. served by server 20. Currently, various alternative browser software applications are commercially available for personal computers and other web-enabled devices, such as a mobile telephone, Internet appliance, personal digital assistant (PDA), etc. The client computer 40 includes a graphical user interface 48 allowing the user to send and receive data to and from the server 20, as generally known in the art for network communication in a web and/or Internet environment.

FIG. 1 further illustrates the data processing system 150 for enhanced medicament-based treatment of disease, which includes a data analysis module 152, a results output module 154, and a data store 156. The data store 156 and modules 152, 154 are shown logically in FIG. 1 for illustrative purposes, without regard to any particular embodiment in one or more software programs. By way of example, the data store 156 may be a database implemented using a suitable commercially available database software program.

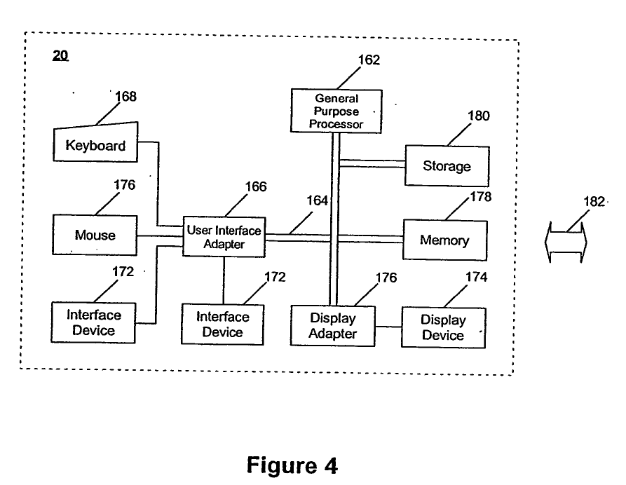

The server 20 of the data processing system 150 further includes various hardware, etc. of a type generally known in the art, as shown in FIG. 4 and discussed further below. As discussed further below, the modules 152, 154 and data store 156 are used in the course of receiving and aggregating data from various users, performing statistical or other analyses on such data to develop information useful correlating at least one patient characteristic with a treatment parameter, and in preparing printed reports, web sites, or other displays of information for use by medicament manufacturers, medicament sales representatives, physicians, other health care professionals, and other interested parties, etc.

In a preferred embodiment, the present invention is implemented via a web site including a plurality of linked web pages. In the example of FIG. 1, the web pages of the web site are served by the HTTP server software 158 of server 20 to one or more client computers 20, and the server receiver data input by users 70 and transmittal via client computers 40. Any suitable database and server architecture may be used, as will be appreciated by those skilled in the art.

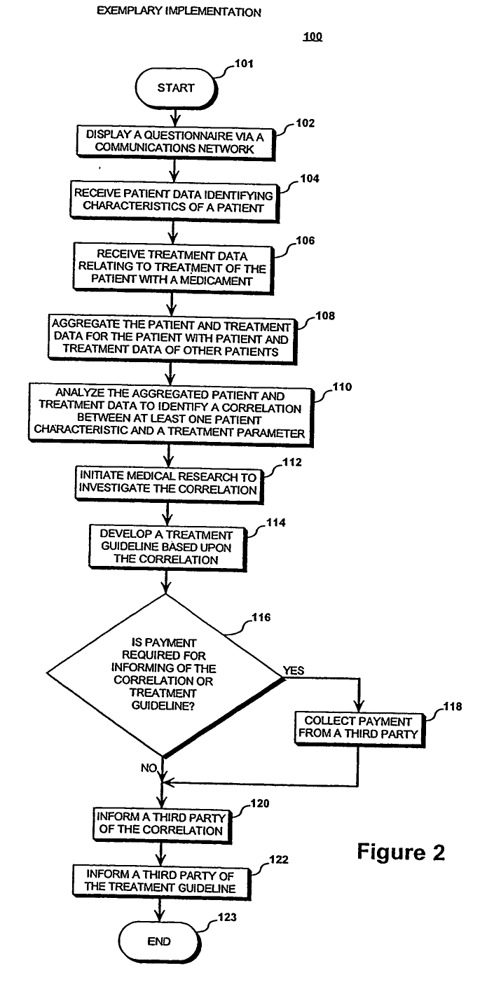

Referring now to FIG. 2, a flow diagram 100 is shown that illustrates an exemplary method in accordance with the present invention. In the example of FIG. 2, it is considered that various human patients, nationally or internationally, are suffering from a certain disease, e.g. diabetes, and have been diagnosed as such by their respective physicians, and have been prescribed a certain medicament, and are following a certain medicament regimen identifying dosage, frequency, etc. according to the respective physician's judgment. Each patient may react differently to a similar prescribed treatment regimen, e.g. experience side effects, respond very favorably, respond moderately favorably, respond unfavorably, be non-responsive, etc. In the absence of the present invention, each of these reactions in various patients would remain unrelated to each other, likely remain unreported, and therefore could not lead to any action on the part of the creator/manufacturer of the drug, e.g. further research, clinical testing, medicament modification/improvement, development of recommended treatment guidelines, etc.

However, in accordance with the present invention, these reactions are reported to a central authority (including the data processing system 150) in the form of data. The data is aggregated and analyzed, e.g. statistically, to provide feedback to interested parties, which may include the creator/manufacturer of the drug, physicians, medical societies, governments, research institutions, consumers, patients, potential patients, data sources having provided data to the system 150, members of the medical community, members of the general public, etc. For example, such data analysis may thereby determine a correlation between such reactions and patient characteristics, such as environmental or physical conditions, characteristics, etc. Such data analysis thereby yields useful information correlating at least one patient characteristic with a treatment parameter, such as concurrent treatments, dosages, length of treatment, medicament selection, formulation, etc. Such data analysis may lead to clinical or other research, and/or development of a corresponding treatment guideline, e.g. specific prescription details, such as smaller or larger dosage, a different length of exposure to the medicament, etc. The results of such research, including any treatment guidelines developed, may then be evaluated and, if warranted, later disclosed to interested parties, e.g. via printed materials distributed by postal service or by hand, or by a communications network, e.g. via a web site, e-mail, etc. Interested parties are thereby quickly made aware of an enhanced treatment—which can then be applied by physicians, etc. to a larger spectrum of patients based upon a better understanding of effects, side effects, etc. of treatment of patients with the medicament. In this manner, patients are better treated and are more satisfied with the beneficial effects of the medicament.

Advantageously, the pro-active approach of the creator/manufacturer of the medicament tends to improve its image with respect to physicians, consumers, sales representatives, etc. Therefore, the enhanced treatment not only better serves the patients, but also tends to better educate physicians, and thereby leads to increased sales of the medicament, to the benefit of the creator/manufacturer of the medicament. Further, the widespread availability of disease, medicament and treatment information, as well as multi-media coordination of promotional efforts, allows a medicament creator/manufacturer to effectively market medicament products, and enables communication of relevant information to individuals that might not otherwise receive such information.

It should be noted that the present invention applies to a variety of living beings, including human beings, animals, plant life, etc., but is discussed below with respect to a human patient. For example, the present invention is particularly well suited to a medicament for treating diabetes, e.g. by treating with a slow-release insulin medicament. Furthermore, the present invention is applicable to a broad range of diseases.

Referring now to FIG. 2, the exemplary method shown starts with display of a questionnaire via a communications network, e.g. via a web page, as shown as steps 101 and 102. The questionnaire is configured to solicit responses provided by an input source via a web or other network interface. By way of example, an input source may include an individual, an organization such as a group of patients, a union, governmental or non-governmental organizations, research institutions, data repositories scattered throughout the Internet or gather from specific databases, etc. It should be noted, therefore, that an input source may be an existing database of patient information from which patient and/or treatment data is actively or passively imported, exported, or otherwise obtained. Particular examples of data sources include a physician, nurse, patient, medicament sales representative, a patient's family member, or a potential patient. Doctors and nurses are particularly important sources of data, as they have close contact with patients, and expertise in evaluating symptoms, prescribing drugs and observing and evaluating the patient's progress over time. The patient, patient's family members, and a potential patient are valuable data sources in that they can provide insight about personal suffering, worries concerning the medication and its eventual side effects, the prevention or prophylaxis of the disease. Sales representatives can offer a commercial-based perspective relating to how physicians view or will likely view a treatment or a medicament, with suggestions as to dosages, presentation, competitors, competing products, medicament price, etc.

A questionnaire posted and freely accessible on a website is exemplary of passive data acquisition in that it may be completed by an individual according to his availability, and upon his own initiative. An example of active data acquisition is the targeted prompting individuals or organizations (e.g. by direct e-mail or postal mail) to respond to specific questions, which responses are then fed to the data store over the information network. The acquisition of data that feeds the data store is either, or both, passive or active, that is, generated by interested parties like individuals or institutions, or searched for by those utilizing the method of the invention.

Next, the method involves receiving patent data identifying characteristics of a patient, as shown at step 104. By way of example, such data may include personal characteristics particular to the patient such as name, age, height, weight, ethnicity, the treated disease, concurrent diseases, concurrent medicaments ingested, inherited genetics, medical history, concurrent diseases, eating, drinking and exercising habits, healthy life habits, personal hygiene, etc. and environmental factors particular to the patient such as employment, family or other stress factors, temperature, pressure, humidity or other climatic aspects air or sound pollution, natural or acquired physical posture, etc. Such patient data may be provided by inputting responses to the questionnaire using the user's 70 client computer 40 (see FIG. 1), using techniques known in the art.

The method further includes receiving treatment data relating to treatment of the patient with a medicament, as shown at step 106. The treatment data may identify treatment parameters (characteristics relating to the treatment plan) or other information. For example, the treatment data may include identification of the medicament (which may include a brand name), the identification may be implicitly specified, e.g. by use of a certain website dedicated for use of that medicament, or may be expressly provided by the data source, e.g. in a response to the questionnaire. Such treatment data may also indicate a treatment regimen, non-medicament portions of a treatment regimen, medicament dosages, frequency, duration of regimen, etc.

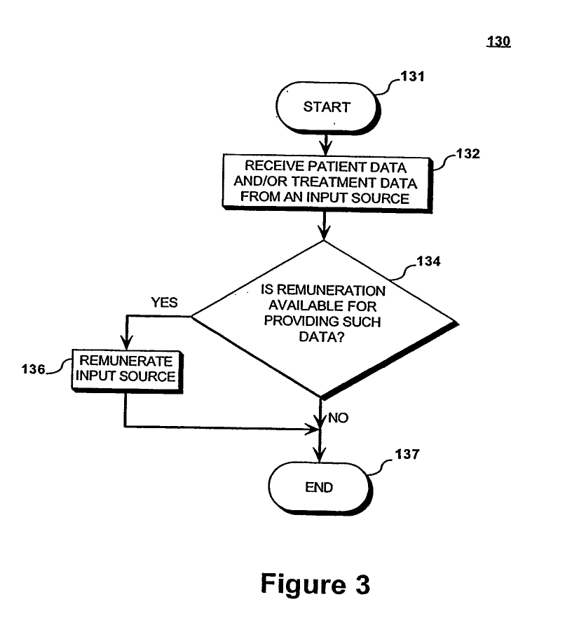

Any such patient and treatment data provided by an input source is transmitted from the client computer 40, etc. via the communications network 60 to the server 20, where it is received and stored in the data store 156 of the server 20. It should be noted that such a server 20 may be operated and/or maintained by a private institution, such as the medicament creator/manufacturer, an independent party administrating the method, etc. Referring briefly to the flow diagram 130 of FIG. 3, step 132 corresponds to steps 103 and 106 of FIG. 2. As shown in FIG. 3, the private institution or other operator of the server 20 may offer remuneration, e.g. money or other compensation/reward, for providing patient and/or treatment data. Accordingly, as shown in FIG. 3, when such data is provided by an input source, it is determined whether remuneration is available for providing such data, i.e. whether it is being offered by the private institution, etc., as shown at step 134. If so, the private institution, etc. remunerates the input source, as shown at step 136, and the method ends, as shown at step 137. This step may be performed by printing and mailing a check to the input source, electronically crediting an account of the input source, issuing a coupon or voucher to the input source, or any other suitable method. If remuneration is not available, the method ends, as shown at step 137. When the method of FIG. 3 ends, the method of FIG. 2 continues.

Referring again to FIG. 2, patient and treatment data provided by an input source for a particular patient in steps 104 and 106 is then aggregated with the patient and treatment data, respectively, of other patients, as shown at step 108. For example, such data may be aggregated with data of other patients that have previously provided patient and treatment data to the server 20.

The aggregated patient and/or treatment data is then analyzed to identify a correlation between at least one patient characteristic and a treatment parameter, as shown at step 10. The processing of the acquired data in this manner is intended to produce useful information that may be used to enhance treatment of the disease, e.g. by identifying areas/topics warranting medical research or other investigation of treatment options, impacts, new medicaments, side effects, etc. To that end, specialists may be used to manually analyze the data, i.e. visually and/or mentally. Alternatively, the analysis may be performed in an automated fashion, e.g. by statistical data analysis or other analysis software. Examples of suitable software are well known in the art. The data analysis is performed by the data analysis module 152 of the server 20.

The analysis of the data may be administered by any suitable entity, such as the creator/manufacturer of the medicament, or another capable entity.

In the embodiment shown, medical research is initiated by an appropriate medical research facility to investigate the correlation, or a selected correlation, identified in step 110, as shown in step 112. Next, a treatment guideline is developed based on the correlation, as shown at step 114. This treatment guideline may be the result of the medical research and/or the identified correlation. For example, if initial research relating to FDA approval of a medicament indicated that medicament X is suitable for treating high blood pressure in humans, and analysis of patient and treatment data indicated that medicament X caused complication Y when the patient had characteristic Z, then a guideline may be developed to caution against prescribing medicament X for a human having high blood pressure if the human has characteristic Z. However, this may lead to medical research, including a new or modified medicament, medicament X′, that is suitable for treating humans having high blood pressure, regardless of whether the human has characteristic Z. Accordingly, medicament X′ would likely become the medicament of choice among knowledgeable physicians and consumers, leading to increased sales of medicament X′, and/or increased sales in medicament X resulting from confidence in physicians and consumers that medicament X is suitable for humans not having characteristic Z, and not known, after analysis of gathered patient and treatment data, to have any other possible complications, e.g. Y′, Y″, Y′″.

In the example of FIG. 2, it is next determined whether payment is required of an interested party, such as a patient, potential patient, physician, etc., in exchange for being informed of the identified correlation, treatment guideline and/or medical research results, as shown at step 116. If a payment is required, payment is collected, e.g. via credit card payment, as shown at step 118, and the method continues. Any suitable payment collection technique may be used. If no payment is required, the method simply continues, as shown at 116. In either case, the party is then informed of the correlation and/or the treatment guidelines, and the method ends, as shown at steps 120, 122 and 123.

The interested party may be informed in any suitable manner. By way of example, the interested party may be informed by advertising in a suitable communications medium, such a print, broadcast radio or television, etc., by oral or printed communications between medicament sales representatives and physicians, nurses and other health care providers, by oral and/or slide presentations at conferences to physicians, nurses and other health care providers, etc. In a preferred embodiment, the interested party is informed by distributing the correlation and/or treatment guideline via the communications network, e.g. via a web page, e-mail, etc., e.g. by transmitting the treatment guideline.

Information obtained from analysis of the patient and treatment data, either directly, or indirectly, e.g. after additional research, is then communicated to interested parties. For example, the information may be communicated to patients, e.g. by mail, e-mail or website, to physicians, e.g. by printed materials and/or oral disclosure by drug representatives or conference speakers, etc. It should be noted that the interested parties may include one or more of the input sources and associated patients that have provided data to the system.

In this manner, analysis of the patient and treatment data can lead to various ways to enhance treatment of a disease. This information may thereby be used to increase sales of the medicament, e.g. by appropriately educating patients, physicians or other interested parties of the availability of such information, the information itself, or information resulting from such information, such as newly developed treatment guidelines. Various issues may be addressed as a result of such information, including the selection of a medicament, and issues relating to the physician, the nurse treating the patient, and the patient. A patient's prescription for the medicament can be modified, as a result of the correlation, guideline and/or medical research, for instance, as to new dosages, new presentation, new composition, etc. With the useful information, the physician can prescribe with greater confidence and precision, aware of effects and side-effects, and with greater focus on certain aspects of a disease. The nurses learn more about the evolution of the treated patient, from their perspective. The patients have access to comprehensive information, via the website displaying such correlations, guidelines and/or medical research results, about the disease itself and the correct way to take the medicament to obtain enhanced effects.

It should be noted that the informing of interested parties allows for more extensive disclosure to physicians of certain aspects of a medicament, including disclosure of comparisons to a competitor's competing medicament product. This leads to increased sales of the medicament, which relates to another aspect of the invention, namely, use of the method described hereinbefore to increase sales of the medicament for the treatment of disease. As discussed above, the use of the method can cause increased sales of the chosen medicament, in large part because patients, potential patients, and physicians, nurses and other health care providers become better informed about the benefits of the medicament, and are provided with extensive information, based on a broad base of patient data and feedback, that lends confidence in prescribing the medicament, recognizing when it may be beneficial, etc. Particularly, the method achieves an increase in sales from 100% to 400% after the use of the invention. The method of the present invention therefore may be used to increase sales of a medicament by at least 100%, at least 200%, at least 300%, and/or at least 400%.

FIG. 4 is a block diagram of a server 20 of the data processing system 150 (see FIG. 1) in accordance with the present invention. As is well known in the art, the server of FIG. 4 includes a general purpose microprocessor (CPU) 162 and a bus 164 employed to connect and enable communication between the microprocessor 162 and the components of the server 20 in accordance with known techniques. The server 20 typically includes a user interface adapter 166, which connects the microprocessor 162 via the bus 164 to one or more interface devices, such as a keyboard 168, mouse 170, and/or other interface devices 172, which can be any user interface device, such as a touch sensitive screen, digitized entry pad, etc. The bus 164 also connects a display device 174, such as an LCD screen or monitor, to the microprocessor 162 via a display adapter 176. The bus 164 also connects the microprocessor 162 to memory 178 and long-term storage 180 (collectively, “memory”) which can include a hard drive, diskette drive, tape drive, etc.

The server 20 may communicate with other computers or networks of computers, for example via a communications channel, network card or modem 182. The server 20 may be associated with such other computers in a local area network (LAN), a wide area network (WAN), intranet, Internet, etc. All of these configurations, as well as the appropriate communications hardware and software, are known in the art.

Software programming code for carrying out the inventive method is typically stored in the memory of the server 20. Accordingly, the server 20 stores in its memory microprocessor executable instructions. These instructions may include a first program for receiving from an input source, via a communications network, patient data identifying characteristics of a particular patient, a second program for receiving from the input source, via the communications network, treatment data relating to treatment of the particular patient with a certain medicament for a certain disease, a third program stored for aggregating said patient data and said treatment data with additional patient data and additional treatment data, respectively, for a plurality of respective patients, a fourth program for analyzing said aggregated patient data and said aggregated treatment data to identify useful information correlating at least one patient characteristic with a treatment parameter.

Optionally the server may store in its memory one or more programs for: developing a treatment guideline for patients having said at least one patient characteristic based upon said useful information; for transmitting the treatment guideline via the communications network; for remunerating an input source of the patient and/or treatment data; for displaying to the input source, via the communications network; a questionnaire soliciting responses from the input source to gather patient and treatment data, analyzing in an automated fashion aggregated data using statistical data analysis techniques; and collecting payment from an interested party in exchange for informing of a correlation and/or a treatment guideline, respectively.

Additionally, computer readable media storing computer readable code for implementing the method steps is provided (see discussion of method steps above).

The present invention is further described in materials attached hereto as Appendix A.

Having thus described particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

### APPENDIX A

METHOD FOR THE ACQUISITION AND USE OF DATA IN OPTIMIZING THE TREATMENT OF DISEASE OR HEALTH DYSFUNCTION OF LIVING BEINGS AND USE OF SUCH METHOD TO INCREASE SALES OF A MEDICAMENT.

The present invention relates to a method where one acquires and uses data concerning a disease or health dysfunction, providing by way of its processing into useful information an optimized treatment for such a disease or health dysfunction. In another aspect of the invention, the use of such a method leads to an increase of sales for a medicament for the treatment of such a disease or health dysfunction.

The invention particularly concerns a method for the optimized treatment of diabetes, and the use of such a method to increase sales of a medicament for treating diabetes.

### BACKGROUND

The treatment of diseases and health dysfunctions relies heavily on the intake of medicaments. The context of treating a disease, viewed in its largest approach, is quite complex.

On one hand, no matter how much money and effort was invested in the development of a medicament, there are always conditions and situations that could not be actually tested, as every human organism is unique in its response to a foreign chemical substance. Many other factors, endogenous and exogenous, might affect a medicinal treatment, like inherited genetic charge, other existing diseases, eating, drinking and exercising habits, healthy life habits, personal hygiene, stress caused by work, family or traffic, climate aspects like temperature, pressure and humidity, air or sound pollution, the natural or acquired physical posture, etc.

On the other hand, independent or mixed commercial factors, which do not necessarily relate to one other, strongly influence the consumption of a medicament, like cost/benefit ratio, the exposure of a certain brand in the specialized media, the familiarity of a prescribing doctor with that drug, the advantages and disadvantages of a medicament with relation to a competitor, the easy access and quick response one obtains from the manufacturer or creator of the medicament, etc.

All these factors are known to those who create, manufacture and sell medicaments. They know how complex it is, in a balanced and efficient manner, to bring a medicament to the market, to disclose it to the medical society, to distributors, to consumers, to sell it, and, most importantly, to treat and to improve the treatment of the disease or dysfunction the medicament is aimed to.

There is a long felt need for ways to combine the improvement in the treatment of diseases or health conditions with improved sales.

The present invention is directed to that target. One advantage of the invention is the capability of extracting useful information from data put together, data that already exists, but would otherwise remain unreported or evaluated only by itself, without taking into account its existence within a larger context. That aspect leverages the sales of a medicament to which the method is applied.

There is no similar invention in the prior art. Although related to medicaments and the use of an information network, the following documents do not disclose, suggest or in any way foresee the present invention: WO0154027, WO0159570, WO0186506, WO0205187, EP1244047, WO0159570, WO0223440, JP2001338055, JP200213289, WO0146888, WO127838, WO0118726, US20020143578, U.S. Pat. No. 6,322,504, US20020082906, US20020147135.

### DESCRIPTION OF THE INVENTION

In one aspect, it provides a method for the acquisition and use of data in optimizing the treatment of disease or health dysfunctions of living beings. In another aspect, it provides the use of that method to increase the sales of a medicament for the treatment of such disease or health dysfunction.

As a simple example, this is how the invention works. A relatively small number of people, in different parts of the world, experience some kind of effect when taking a medicament for a certain health dysfunction. In the absence of this invention, these events would probably remain unrelated to each other, and it would not lead to any action on the part of the creator/manufacturer of the drug. But, with the invention, that data is directed through the Internet to a database, whose administrators are then able to process the data, for instance statistically, and determine that the reported events effect, for instance, patients sharing similar specific organic conditions, or race characteristics. That acquired data which became information leads to research, and specific prescription details—such as smaller or larger dosage, or a different time exposure to the drug—are evaluated and later disclosed to the public on the site of that company. The public is then quickly made aware of the optimized treatment—which the can be applied by doctors to a larger spectrum of patients based on an amplified knowledge about effects, patients are more satisfied with the beneficial effects of the medicament. On the commercial side, the pro-active attitude of the company improves its image before consumers, sales representatives use the new information as a tool to inform doctors. Therefore, the optimized treatment, which is the outcome of the use of acquired data, affects and improves sales of the medicament.

The method of the invention comprises the steps of:
A. acquiring data from input source A through an information network X;
B. feeding acquired data to a database B;
C. processing said data in the database, transforming it into useful information;
D. using said processed data to improve issues related to said disease or health dysfunction treatment;
E. providing output information related to said improvement thought an information network.

The method of the invention applies to living beings, humans, animals or vegetal. A particular embodiment relates to optimizing the treatment of human diseases or health dysfunctions.

The attached FIG. 1 illustrates the method of the invention.

A is any source of data related to the a disease or health dysfunction, for instance individuals or organizations like groups of patients, unions, non-governmental organizations, government organizations, research institutions, data scattered on the Internet or gathered in specific databases, etc.

Particular examples of data sources are individuals, like a doctor, a nurse, a patient, a sales representative, a patient's family member, or potential patient. Doctors and nurses are important sources of data, as they are the ones with closer contact with patients, the former evaluating symptoms and prescribing drugs, the latter with a day-to-day expert eye on the evolution of the patient. The patient, members of his family, or a potential patient who are another important source of data, providing insight about personal suffering, worries concerning the medication and its eventual side effects, the prevention or prophylaxis of the disease. Sales representatives bring a commercial view on how the doctors view a treatment or a medicament, with suggestions as to dosages, presentation, competitors, price, etc.

A preferential embodiment of the invention relates to optimizing the treatment of diabetes in human beings. Particularly it concerns a treatment with slow release insulin.

An information network X is the tool utilized to acquire data in the method of the invention. The main alternative of information network X is the Internet, but also a more limited network is also encompassed by the invention, for instance an intranet in a hospital, or any configuration allowing data to flow between points of access. The alternative ways of acquiring data is not a limitation of the invention, provided it can reach the database B. Typical alternatives are a simple e-mail or answers to a questionnaire made available over the network by those utilizing the method of the invention.

The acquisition of data that feeds database B is either, or both, passive or active, that is, generated by third parties like individuals or institutions, or searched for by those utilizing the method of the invention. An example of passive acquisition is a questionnaire made available on an Internet site, which is filled in by an individual according to his availability. An example of active acquisition is prompting individuals or organizations to respond to specific questions, which then come back to feed database B over the information network.

The acquired data is either purchased from or provided for free by the input source A. In another alternative, input source A is rewarded for providing data.

The acquired data is fed to database B either directly via software or equivalent electronic tool, or manually input.

The processing of the acquired data aims at obtaining useful information to optimize the treatment of a disease or health dysfunction. To that end, specialists and/or electronic tools like statistical software, assess the data to extract useful information. The processing of the data is administered by an entity which can be the creator, or the manufacturer of the medicament, or any other capable entity. Particularly, the administrator is a private institution whose interests relate to the specific disease or health dysfunction the method is applied to.

The outcome of processing the data in item C to obtain useful information leads to different ways of optimizing the treatment of a disease or health dysfunction. Different issues are addressed, typically the medicament itself, the doctor and the nurse treating the patient and the patient. The medicament can be modified, for instance as to new dosages, new presentation, new composition. With the useful information, the doctor can prescribe with more focus, aware of effects and side-effects, more specifically directed to certain aspects of a disease or dysfunction. The nurses get to know more about the evolution of the treated patient, from their perspective. The patients also get more comprehensive information about the disease itself and the correct way to take the medication, so as to obtain optimized effects.

The improvement of step D of this method can also lead to more commercial attitudes, such as more disclosure to the doctors of certain aspects of a medicament, less known or underestimated by them, or in comparison to a competitor product.

Step D of this method is not limited to any specific action or attitude, as it is based on useful information that remained unknown until data was collected and analyzed as provided by steps A, B and C.

In step E of the method, information about the improvement in the issues related to the treatment of the disease or health dysfunction is made available to interested parties, for instance the same input resource A or any other. This information can be freely provided, or sold at a price. In an alternative, the data transformed in useful information, in step C can also be made available to interested parties. The way to make the information available to third parties is through an information network, such as the Internet or any other.

Another aspect of the invention is the use of the method described hereinbefore to obtain increase in the sales of the medicament for the treatment of a chosen disease or health dysfunction. As already mentioned, the achievement of the various steps of the invention have a beneficial effect on the sales of the chosen medicament. Particularly, the method achieves an increase in sales from 100% to 400% after the use of the invention.

The use of the method to increase sales applies particularly to the increase in sales of a medicament for the treatment of diabetes, more specifically with slow release of insulin.

It can be readily perceived from the description of the invention that many alternatives can be employed without departing from the scope of the invention or equivalents thereof, as defined in the attached claims.

METHOD FOR THE ACQUISITION AND USE OF INFORMATION TOWARDS OPTIMIZING THE TREATMENT OF DISEASE OR HEALTH DYSFUNCTION OF LIVING BEINGS comprising the steps of:

A. acquiring data from input source through an information network;

B. feeding acquired data to a database;

C. processing said data in the database, achieving its intelligence into useful information;

D. using said processed data to improve issues related to said disease or health dysfunction treatment;

E. providing output information related to said improvement thought an information network.

Method of claim 1 wherein said living beings are human beings.

Method of claim 1 wherein said health dysfunction is diabetes.

Method of claim 1 wherein said input source is one or more of the following objects: physicians, nurses, sale representatives, patients, patient's family members, and potential patients.

Method of claim 1 wherein the data acquired in step A is either purchased from or voluntarily provided by said input source.

Method of claim 1 wherein said input source are answers to questions presented to the input source on the interface of said information network.

Method of claim 1 wherein the processing in step C is achieved mechanically and/or by direct analysis by persons.

Method of claim 1 wherein said improvement in step D concerns specifically the medicament to treat said disease or dysfunction.

Method of claim 1 wherein improvement in step D concerns specifically the patient that takes the medicament to treat said disease or dysfunction.

Method of claim 1 wherein improvement in step D concerns specifically the physician that prescribes the medicament to treat said disease or dysfunction.

Method of claim 1 wherein improvement in step D concerns specifically the nurse that takes care of patients take the medicament to treat said disease or dysfunction.

Method of claim 1 wherein said information network is the Internet.

Method of claim 1 wherein the input source is rewarded for providing information related to said disease or health dysfunction.

Method of claim 1 wherein the improvement in step D concerns better disclosure related to the medicament to treat said disease of health dysfunction.

Method of claim 1 wherein step D is administrated by a private institution.

Method of claim 1 wherein the output information in step E is either free or sold to interested consumers.

Use of the method in any of the preceding claims to increase sales of a medicament for said disease or health dysfunction.

Use of the method of claim 17 wherein said medicament concerns slow release of insulin in the treatment of diabetes.

Use of the method in claims 17 or 18 to increase sales of said medicament in at least 100%.

Use of the method in claims 17 or 18 to increase sales of said medicament in at least 200%.

Use of the method in claims 17 or 18 to increase sales of said medicament in at least 300%.

Use of the method in claims 17 or 18 to increase sales of said medicament in at least 400%.

What is claimed is:

1. A method for enhancing medicament-based treatment of disease, the method comprising:
receiving from an input source, via a communications network, patient data identifying characteristics of a particular patent;
receiving from the input source, via the communications network, treatment data relating to treatment of the particular patient with a certain medicament for a certain disease;
aggregating said patient data and said treatment data with additional patient data and additional treatment data, respectively, for a plurality of respective patients;
analyzing said aggregated patient data and said aggregated treatment data to identify useful information correlating at least one patient characteristic with a treatment parameter; and
developing a treatment guideline for patients having said at least one patient characteristic based upon said useful information.

2. The method of claim 1, further comprising:
transmitting said treatment guideline via said communication network.

3. The method of claim 1, wherein said patient comprises a human being.

4. The method of claim 1, wherein said disease is diabetes.

5. The method of claim 1, wherein said input source is selected from the group consisting of: a physician, a nurse, a medicament sales representative, a patient, a patient's family member, a potential patient and combinations of two or more thereof.

6. The method of claim 1, further comprising:
remunerating said input source for providing said patient data.

7. The method of claim 1, further comprising:
remunerating said input source for providing said treatment data.

8. The method of claim 1, further comprising:
displaying to said input source, via said communications network, a questionnaire soliciting responses from said input source to gather said patient data and said treatment data.

9. The method of claim 1, wherein said analyzing is performed in an automated fashion.

10. The method of claim 9, wherein said analysis is performed in an automated fashion by computer software employing statistical data analysis techniques.

11. The method of claim 1, wherein said analyzing is performed manually.

12. The method of claim 1, wherein said treatment guideline specifies a certain medicament.

13. The method of claim 1, wherein said treatment guideline specifies a certain medicament intake regimen.

14. The method of claim 1, wherein said treatment guideline specifies instructions to one selected from the group consisting of: a physician, a nurse, a medicament sales representative, a patient, a patient's family member, a potential patient and combinations of two or more thereof.

15. The method of claim 1, further comprising informing an interested party of said useful information.

16. The method of claim 15, further comprising collecting payment from said interested party in exchange for said informing.

17. The method of claim 1, further comprising informing an interested party of said treatment guideline.

18. The method of claim 17, further comprising collecting payment from an interested party in exchange for said informing.

19. The method of claim 1, wherein said analysis is performed by a private institution.

20. The method of claim 1, further comprising advertising in a communications medium an ability to create said treatment guideline.

21. The method of claim 1, further comprising advertising availability of said treatment guideline for treatment of said disease using said medicament.

22. The method of claim 1, wherein said medicament is capable of providing a slow release of insulin in the treatment of diabetes in a living being.

23. A method for enhancing medicament-based treatment of disease, the method comprising:
receiving from an input source, via a communications network, patient data identifying characteristics of a particular patient;
receiving from the input source, via the communications network, treatment data relating to treatment of the particular patient with a certain medicament for a certain disease;
aggregating said patient data and said treatment data with additional patient data and additional treatment data, respectively, for a plurality of respective patients;
analyzing said aggregated patient data and said aggregated treatment data; and
identifying a statistically significant correlation between at least one patient characteristic and a treatment parameter.

24. A method for enhancing medicament-based treatment of disease, the method comprising:
receiving from an input source, via a communications network, patient data identifying characteristics of a particular patient;
receiving from the input source, via the communications network, treatment data relating to treatment of the particular patient with a certain medicament for a certain disease;
aggregating said patient data and said treatment data with additional patient data and additional treatment data, respectively, for a plurality of respective patients;
analyzing said aggregated patient data and said aggregated treatment data; and
identifying a statistically significant correlation between at least one patient characteristic and an effectiveness of said medicament for treating said disease.

25. The method of claim 24, further comprising initiating medical research to investigate said correlation.
